(19) United States
(12) Patent Application Publication
(10) Pub. No.: US 2010/0311998 A1
(43) Pub. Date: Dec. 9, 2010

(54) CHELATING AGENT BASED IONIC LIQUIDS, A PROCESS TO PREPARE THEM, AND THEIR USE

(75) Inventors: Cornelia Adriana De Wolf, Eerbeek (NL); Boris Kuzmanovic, Serbia (NL); Johanna Hendrika Bemelaar, Arnhem (NL); Cornelis Johannes Govardus Van Strien, Elst (NL); Martin Heus, Arnhem (NL); Antoon Ten Kate, Arnhem (NL)

Correspondence Address: KENYON & KENYON LLP, ONE BROADWAY, NEW YORK, NY, 10004, US

(73) Assignee: AKZO NOBEL N.V., ARNHEM (NL)

(21) Appl. No.: 12/671,667

(22) PCT Filed: Jul. 31, 2008
(86) PCT No.: PCT/EP2008/060066

§ 371 Date: Apr. 27, 2010

Related U.S. Application Data
(60) Provisional application No. 60/954,419, filed on Aug. 7, 2007.

(30) Foreign Application Priority Data
Aug. 2, 2007 (EP) 07113716.0

(51) Int. Cl. C07F 5/06; C07C 229/26; C07F 11/00; C07C 229/46

(52) U.S. Cl. 556/27; 562/565; 556/63; 562/507

(57) ABSTRACT

The present invention relates to ionic liquids of the formula: (M^{m+})ₓH⁺_{y}nN⁺R₁R₂R₃R₄Z—N(—CHXCOO⁻)ₚY₂₋ₚ or (M^{m+})ₓH⁺_{y}nP⁺R₁R₂R₃R₄Z—N(—CHXCOO⁻)ₚY₂₋ₚ wherein any one of R₁ to R₄ independently represents a hydrogen, alkyl, cycloalkyl, aryl or aralkyl group that may be unsubstituted or substituted with one or more groups selected from OH, Cl, Br, F, I, phenyl, NH₂, CN, NO₂, an ether group, COOR₅, CHO, COR₅ or OR₅, wherein R₅ is an alkyl or cycloalkyl group, and wherein two of the groups R₁ to R₄ together with the N atom may form a heteroaromatic or heterocyclic group such as a pyridinium, pyrrolidinium or imidazolium group; the Z—N(—CHXCOO⁻)pY₂₋ₚ is the anion derived from a chelating agent, wherein Z is a group selected from hydrogen, alkyl, an alkyl group optionally substituted with one or more carboxylate groups, hydroxyl groups and/or optionally containing one or more ether functionalities, or Z is a group of the formula —CH₂—CHR₆—R₇—N—(CH₂COO⁻)₂, R₆ is hydrogen or a C₁-C₃ alkyl group, R₇ is a single bond, a C₁-C₃ alkylene group or one or more groups of the formula —N(CH₂COO⁻)—CH₂—CHR₆—R₈—, R₈ is a single bond or a C₁-C₃ alkylene group, p is 2 or 1, X is H, an aryl group that may be unsubstituted or substituted with an OH and/or CH₃ substituent, or CH₂COO⁻, and when p is 1, Y is H, a benzyl group that may be unsubstituted or substituted with an OH and/or CH₃ group, or a phenyl group that may be unsubstituted or substituted with an OH and/or CH₃ substituent, M^{m+} is a m-valent metal cation, n is 1 or higher, x is from 0 to 5, y is from 0 to 3, the total positive charge of (M^{m+})ₓ, H⁺_{y}, and nN⁺R¹R²R³R⁴ or nP⁺R₁R₂R₃R₄ equals the negative charge of the Z—N(—CHXCOO⁻)ₚY₂₋ₚ anion, wherein at least one of the following conditions is fulfilled: i) the chelating agent has a connectivity index ¹X/³X of below 2.17, ii) at least two different cations are present in the salt, whereby a proton cation does not count as one of the at least two, iii) at least one cation is a bivalent, trivalent, tetravalent or pentavalent cation, and having a melting point below 120° C. The invention further relates to a process to prepare them and their use.

The present invention relates to ionic liquids containing an anion derived from a chelating agent or a derivative thereof as anion, a process to prepare them, and the use thereof.

Ionic liquids are non-volatile salts with a melting point below 120° C. Many are liquid even at room temperature and they represent a relatively new class of solvents.

It is known that, in general, ionic liquids may be used in many applications, e.g. as reaction solvents, extraction solvents, electrolytes, catalysts, heat exchange fluids, and as additives in coatings.

A number of examples of the use of ionic liquids are disclosed for example on Merck's and Iolitec's web pages: www.ionicliquids-merck.de and www.iolitec.com (dated Feb. 3, 2006). Many ionic liquids known today are based on imidazolium cations, or trifluoromethyl sulfonylimide anions, which are quite expensive and the latter of which carry the risk of very dangerous HF being formed as a decomposition product of fluorine-containing ionic liquids.

Chelation is the process of reversible binding (complexation) of a chelating agent (also known as a sequestering agent or complexing agent) to a metal ion, forming a metal complex, the chelate. The term is generally reserved for complexes in which the metal ion is bound to two or more atoms of the chelating agent.

Many chelating agents contain an amino N,N-diacetic acid group (a group Z—N—(CH₂—COOH)₂). The most common chelating agent known is EDTA (ethylenediamine N,N,N′,N′-tetra-acetic acid).

U.S. Pat. No. 3,026,265 discloses the preparation of an alkanolamine salt of EDTA and the use thereof in a detergent composition. It is indicated that if the water is evaporated, pure alkanolamine trisalt of EDTA can be acquired (in the solid form). In the examples the triethanolamine, diethanolamine, monoethanolamine, and triisopropanolamine trisalts of EDTA are specifically disclosed. It is not acknowledged that the alkanolamine trisalt of EDTA is an ionic liquid.

JP 2002-356464 discloses a high-purity amine salt of aminopolycarboxylic acid produced by neutralizing the acid with an amine derivative. The amine compound is specifically said to be ammonia, triethylamine, triethanolamine, ethylenediamine, and diethylenetriamine. The aminopolycarboxylic acids used in the examples are EDTA, PDTA, DTPA, ADA, EDDS, ASDA, MGDA, and GLDA. In the examples an aqueous solution of the ammonium salts is prepared. The pure ammonium salts (i.e. salts not solved in water) are not disclosed.

WO 2005/019137 discloses a method for extracting impurities using ionic liquids based on the general formula [K]ₙ⁺[A]ⁿ⁻. The cation Kₙ⁺ of the ionic liquid can be an ammonium cation of the general formula N⁺R₁R₂R₃R₄. The anion can be a carboxylate of the formula [Rf—COO]⁻ but is not specified to be a chelating agent derived anion (i.e. a compound comprising an aminodiacetic acid group).

WO 2005/097731 discloses ionic liquid comprising an anion and a cation wherein the cation is an ammonium ion containing a protonated nitrogen atom. As the anion that can be used ethylenediamine tetra-acetate is mentioned. The specific compounds disclosed in this application are the monoammonium salt ethanolammonium EDTA, diethanolammonium EDTA, N-butyldiethanolammonium EDTA, N,N-dimethylethanolammonium EDTA, N-methylethanolammonium EDTA, and N,N-di(methoxyethyl)ammonium EDTA. Also disclosed is a process to prepare the ionic liquid encompassing the steps of providing the amine and neutralizing this compound with an acid. Finally, the document discloses the use of the ionic liquids in/as a solvent for enzyme-catalyzed reactions.

It was found that the ionic liquids as disclosed in WO 2005/097731 containing EDTA as the anion are only ionic liquids at a relatively high temperature (i.e. they have a relatively high melting point), which makes them less suitable for a number of applications and besides makes them susceptible to thermal degradation.

The aim of the present invention is to find new ionic liquids that are easy to prepare in a one-step synthesis, are composed of widely available raw material, are relatively cheap and non-toxic, can be readily biodegradable depending on the starting material, and combine the metal complexing capacity of the chelating agent (and inherently the metal releasing capacity as well) with the properties of ionic liquids at a lower temperature, so that they are applicable in more applications without suffering from thermal degradation.

More specifically, the present invention aims to develop ionic liquids that can be beneficially used in applications where an ionic liquid that possesses the specific characteristics of a chelating agent is desired.

The present invention now provides ionic liquids of the formula:

(M^{m+})ₓH⁺_{y}nN⁺R₁R₂R₃R₄Z—N(—CHXCOO⁻)ₚY₂₋ₚ or

(M^{m+})ₓH⁺_{y}nP⁺R₁R₂R₃R₄Z—N(—CHXCOO⁻)ₚY₂₋ₚ

wherein
any one of R₁ to R₄ independently represents a hydrogen, alkyl, cycloalkyl, aryl or aralkyl group that may be unsubstituted or substituted with one or more groups selected from OH, Cl, Br, F, I, phenyl, NH₂, CN, NO₂, an ether group, COOR₅, CHO, COR₅ or OR₅, wherein R₅ is an alkyl or cycloalkyl group, and wherein two of the groups R₁ to R₄ together with the N atom may form a heteroaromatic or heterocyclic group such as a pyridinium, pyrrolidinium or imidazolium group;
Z—N(—CHXCOO⁻)ₚY₂₋ₚ is the anion derived from a chelating agent, wherein Z is a group selected from hydrogen, alkyl, an alkyl group optionally substituted with one or more carboxylate groups, hydroxyl groups and/or optionally containing one or more ether functionalities, or Z is a group of the formula —CH₂—CHR₆—R₇—N—(CH₂COO⁻)₂, R₆ is hydrogen or a C₁-C₃ alkyl group, R₇ is a single bond, a C₁-C₃ alkylene group or one or more groups of the formula —N(CH₂COO⁻)—CH₂—CHR₆—R₈—, R₈ is a single bond or a C₁-C₃ alkylene group, p is 2 or 1, X is H, an aryl group that may be unsubstituted or substituted with an OH and/or CH₃ substituent, or CH₂COO⁻ and when p is 1, Y is H, a benzyl group that may be unsubstituted or substituted with an OH and/or CH₃ group, or a phenyl group that may be unsubstituted or substituted with an OH and/or CH₃ substituent,
M^{m+} is a m-valent metal cation,
n is 1 or higher, x is from 0 to 5, y is from 0 to 3, preferably of from 0 to 1,
the total positive charge of (M^{m+})ₓ,H⁺_{y}, and nN⁺R₁R₂R₃R₄ or nP⁺R₁R₂R₃R₄ equals the negative charge of the Z—N(—CHXCOO⁻)ₚY₂₋ₚ anion,
wherein at least one of the following conditions is fulfilled
(i) the chelating agent has a connectivity index ¹X/³X of below 2.17,
(ii) at least two different cations are present in the salt, whereby a proton cation does not count as one of the at least two,
(iii) at least one cation is a bivalent, trivalent, tetravalent or pentavalent cation,
   and having a melting point below 120° C.

Connectivity index ⁱX represents the i^{th} order connectivity of a chelating agent in its acid form. The connectivity index is frequently used in QSPR/QSAR studies (quantitative structure-property-activity relationship). In these studies, the property or activity of a given substance is related to its structure. Connectivity indices are computational entities to characterize the chemical structure and do not have a real physical meaning as such but can be given a physical interpretation. ¹X is said to give a measure of branching and ³X a measure of branching adjacency, so ¹X/³X could be interpreted as a combined measure, indicating the molecular asymmetry. Further information about computing connectivity and their use to relate properties and activities to structures can be found in M. Randic, “The connectivity index 25 years after”, Journal of Molecular Graphics and Modelling, 20 (2001), 19-35 and L. H. Hall, L. B. Kier, “Issues in representation of molecular structure, The development of molecular connectivity”, Journal of Graphics and Modelling, 20 (2001), 4-18.

For this application ¹X and ³X were calculated using the software tool Molecular Modeling Pro, version 4.1.1 (2001) published by ChemSW®.

It was found that a number of compounds of the formula (M^{m+})ₓH⁺_{y}nN⁺R₁R₂R₃R₄Z—N(—CHXCOO⁻)ₚY₂₋ₚ or (M^{m+})ₓH⁺_{y}nP^{+R}₁R₂R₃R₄Z—N(—CHXCOO⁻)ₚY₂₋ₚ were solids until a relatively high temperature up to and including 120° C. These chelating agent based salts not being ionic liquids or being ionic liquids only at a relatively high temperature were determined to be those compounds wherein (i) the chelating agent had a connectivity index ¹X/³X of above 2.17, (ii) all the cations were the same nN⁺R₁R₂R₃R₄ group or nP⁺R₁R₂R₃R₄ group or a proton, and (iii) all the cations were monovalent.

Examples of chelating agents that have a connectivity index ¹X/³X of above 2.17 in the acid form are EDTA and NTA.

The ionic liquids of the invention can be used in many applications. The present invention provides the use of the ionic liquids in applications where both the beneficial properties of ionic liquids and the metal complexing or metal releasing properties of the chelating agent are useful. More specifically, the invention provides the use thereof as reaction solvents, extraction solvents, electrolytes, catalysts, heat exchange fluids, and additives in coatings.

The invention also provides a method to prepare the new ionic liquids. The method comprises mixing of a chelating agent or a salt thereof, and when a metal cation containing ionic liquid is desired, a metal salt of the chelating agent, with an amine or phosphine or the salt, preferably the hydroxyl salt, hydrogen carbonate (HCO₃⁻), methylcarbonate (CH₃OCOO⁻) or carbonate (CO₃²⁻) salt, of an ammonium cation or phosphonium cation in a solvent and subsequently removing the solvent and the other compounds that are formed.

The metal chelate starting material can be formed by the reaction between the chelate, preferably in the acid form, and the metal salt, preferably the hydroxide or oxide.

The other compounds formed in one embodiment are the reaction product of the cation released by the chelating agent and the anion released by the amine, phosphine or the salt thereof. The other compound may for example be CO₂, water or methanol.

It should be understood that only salts are formed when the ammonium cation or phosphonium cation of the ionic liquid, as the case may be, is not of such a nature that the proton will significantly move to the chelating agent, or in other words, the nN⁺R₁R₂R₃R₄ or nP⁺R₁R₂R₃R₄ group wherein one of R₁ to R₄ is a hydrogen atom should not have a more acidic pKa than Z—N(—CHXCOO⁻)ₚY₂₋ₚ in its protonated form.

In the method the chelating agent may be used in the acidic or (partial) salt form.

In a preferred embodiment the solvent used is water. In another preferred embodiment the chelating agent is added in the form of the acid.

The present invention also relates to ionic liquids wherein not only an ammonium or phosphonium cation but also a metal cation is present as a cation. M^{m+} is a m-valent metal cation and the total positive charge of (M^{m+})ₓ and nN⁺R₁R₂R₃R₄ or nP⁺R₁R₂R₃R₄ equals the negative charge of the Z—N(—CHXCOO⁻)ₚY₂₋ₚ anion. This group of ionic liquids is especially suitable for application where the presence of a metal cation is desirable.

As indicated before, preferred chelating agents are those chelating agents of which the connectivity index ¹X/³X is below 2.17, as they provide better flexibility in forming an ionic liquid (e.g. they are also found to be ionic liquids in combination with only monovalent cations).

Though applicant does not intend to give a complete list of chelating agents, the following chelating agents were calculated to have a connectivity index ¹X/³X of below 2.17:

| Chelating agent | Full chemical name | ¹X/³X |
|---|---|---|
| ASDA | Aspartic acid-N,N-diacetic acid | 2.06 |
| CDTA | 1,2-diaminocyclohexane-N,N,N′,N′-tetra- | 1.81 |
| | acetic acid | |
| DTPA | Diethylenetriamine-N,N,N′,N″,N″-penta- | 2.08 |
| | acetic acid | |
| EDDHA | Ethylenediamine-N,N′,diorthohydroxy- | 1.46 |
| | phenylacetic acid | |
| EDDHMA | Ethylene diamine-N,N′,diorthohydroxy- | 1.45 |
| | paramethylphenylacetic acid | |
| EDDS | Ethylenediamine-N,N′-disuccinic acid | 1.91 |
| GLDA | Glutamic acid-N,N-diacetic acid | 2.01 |
| HBED | N,N′-bis(2-hydroxybenzyl)-ethylenediamine- | 1.58 |
| | N,N′-diacetic acid | |
| HEDTA | N-Hydroxyethylethylenediamine, N,N′,N′- | 2.16 |
| | triacetic acid | |
| IDS | Imino-N,N-disuccinic acid | 1.91 |
| MGDA | Methylglycine-N,N-diacetic acid | 1.90 |
| PDTA 1,2 | Propylene 1,2-diamine N,N,N′,N′tetra-acetic | 2.06 |
| | acid | |
| TTHA | Triethylenetetraamine-N,N,N′,N″,N′″,N′″- | 2.03 |
| | hexaacetic acid | |

In a preferred embodiment the chelating agent of the formula Z—N(—CH₂COO⁻)₂ (i) contains at least 1 chiral C atom, (ii) contains a nitrogen atom containing three different groups bound thereto, and/or (iii) contains three or more nitrogen atoms. It was found that using chelating agents satisfying at least one of the three above criteria had on average a lower melting point than chelating agents that do not satisfy any of them.

More preferably, the Z—N(—CHXCOO⁻)ₚY₂₋ₚ anion has a molecular weight of 200 g/mol or higher. In yet another more preferred embodiment n is 2 and X is H (i.e. Z—N(—CH₂COO⁻)₂), even more preferably the anion Z—N(—CHXCOO⁻)ₚY₂₋ₚ is the anion or partially deprotonated anion of HEDTA (hydroxyethylethylenediamine triacetic acid), DTPA (diethylenetriamine penta-acetic acid), MGDA (methylglycine diacetic acid), or GLDA (glutamic acid diacetic acid).

In a preferred embodiment N⁺R₁R₂R₃R₄ is the ammonium cation of a commercially available C₁-C₂₆ alkyl- or C₁-C₂₆ alkanol-substituted amine, such as tetra-alkylammonium, trialkylammonium, trialkanolammonium. Even more preferred are tetra-alkylammonium or trialkylammonium cations. Most preferably, the cation is the tetrabutylammonium, triethylammonium or choline cation.

In a preferred embodiment P⁺R₁R₂R₃R₄ is the phosphonium cation of a commercially available C₁-C₂₆ alkyl- or C₁-C₂₆ alkanol-substituted phosphine, such as tetra-alkylphosphonium, trialkylphosphonium, trialkanolphosphonium. More preferably, the cation is the tetrabutylphosphonium cation.

In a more preferred embodiment the cation N⁺R₁R₂R₃R₄ or P⁺R₁R₂R₃R₄ is selected from the group of the ammonium cations of C₁-C₄ alkyl- or C₁-C₄ alkanol-substituted amines, such as tetra-alkylammonium, trialkylammonium, trialkanol-ammonium or the phosphonium cations of a C₁-C₄ alkyl- or C₁-C₄ alkanol-substituted phosphine, such as tetra-alkylphosphonium, trialkylphosphonium, trialkanolphosphonium.

In another preferred embodiment the metal cation M^{m+} is selected from the group of the cations of chromium, aluminium, copper, lithium, iron, zinc, nickel, titanium, and tin. Even more preferred are the cations of chromium, aluminium, and copper.

In an even more preferred embodiment the ionic liquid is the ammonium or phosphonium salt of GLDA or HEDTA. Most preferred is the tetraammonium or tetraphosphonium salt of GLDA.

The ionic liquids of the invention in one embodiment are free of solvent. Free of solvent means that the ionic liquids contain less than 15% solvent on the total weight of the ionic liquid. Preferably, they contain less than 10 wt % of solvent, more preferably less than 8 wt %, even more preferably less than 5 wt %.

Solvent may mean an organic solvent or water but does not include the cation of the ionic liquid that may be derived from an organic solvent, such as the cation of an amine solvent needed to balance the negative charge of the chelating agent anion.

The invention is illustrated by, but not limited to, the following examples:

### EXAMPLE 1

### Preparation of GLDA-(TBP)₄

3.42 grams of glutamic acid diacetic acid (GLDA, 38.5 wt % in H₂O, Dissolvine® ex AKZO Nobel, 5 mmol) in a 50 ml round-bottom flask were mixed with 13.82 grams of tetrabutylphosphonium hydroxide (TBPH, 40 wt % in H₂O, FLUKA, 20 mmol) in order to replace all acidic protons with TBP cations. The solution was stirred at room temperature until a clear and homogeneous solution was obtained. Subsequently, the solution was subjected to a combination of evaporation in a Rotavap at a temperature of about 60° C. and minimal pressure of about 40 mbar followed by storage in a vacuum oven for 1 week at 50° C. until no further evaporation of the solvent was observed. The remaining product was a viscous liquid and was confirmed to be GLDA tetra TBP by ¹H-NMR.

### EXAMPLE 2

### Preparation of GLDA-(TBP)₂

The procedure was as in Example 1, but with 3.42 grams glutamic acid diacetic acid (GLDA, 38.5 wt % in H₂O, Dissolvine® ex AKZO Nobel, 5 mmol) and 6.91 grams tetrabutylphosphonium hydroxide (TBPH, 40 wt % in H₂O, FLUKA, 10 mmol). In this example only half of the acetic protons are replaced by the TBP cations.

### EXAMPLE 3

### Preparation of HEDTA-(TBA)₃

The procedure was as in Example 1, but with 1.41 grams hydroxyethylenediamino-triacetic acid (HEDTA, 99 wt %, AKZO Nobel, 5 mmol) and 9.73 grams tetrabutyl-ammonium hydroxide (TBAH, 40 wt % in H₂O, ACROS, 15 mmol).

### EXAMPLE 4

### Preparation of DTPA-(Tri Ethanol Amine)₅

The procedure was as in Example 1, but with 1.98 grams diethylenetriaminopenta-acetic acid (DTPA, 99.5 wt %, Dissolvine® ex AKZO Nobel, 5 mmol), 3.73 grams tri-ethanolamine (99.9 wt %, Baker, 25 mmol), and 10 grams H₂O.

### EXAMPLE 5

### Preparation of GLDA-(TBP)₂(TBA)₂

The procedure was as in Example 1, but with 3.42 grams glutamic acid diacetic acid (GLDA, 38.5 wt % in H₂O, AKZO Nobel, 5 mmol), 6.91 grams tetrabutyl-phosphonium hydroxide (TBPH, 40 wt % in H₂O, FLUKA, 10 mmol), and 6.49 grams tetrabutylammonium hydroxide (TBAH, 40 wt % in H₂O, ACROS, 10 mmol). In this example half of the acetic protons are replaced by TBP cations and the other half by TBA cations.

### EXAMPLE 6

### Preparation of HEDTA-(TBP)_{1.5} (Tri Ethanol Amine)_{1.5}

The procedure was as in Example 1, but with 1.41 grams hydroxyethylenediamino-triacetic acid (HEDTA, 99 wt %, AKZO Nobel, 5 mmol), 5.18 grams tetrabutyl-phosphonium hydroxide (TBPH, 40 wt % in H₂O, FLUKA, 7.5 mmol), and 1.12 grams triethanolamine (99.9 wt %, Baker, 7.5 mmol). In this example half of the acetic protons are replaced by TBP cations and the other half by triethylamine cations.

### EXAMPLE 7

### Preparation of Cr(III)-EDTA-TBP

The procedure was as in Example 1, but with 2.21 grams Chromium(III) hydrogen ethylenediaminotetra-acetic acid (Cr(III)H-EDTA, 81.1 wt %, AKZO Nobel, 5 mmol) and 3.46 grams tetrabutylphosphonium hydroxide (TBPH, 40 wt % in H₂O, FLUKA, 5 mmol). The product was a viscous liquid at 120° C.

### EXAMPLE 8

### Preparation of Al-DTPA-(TBP)₂

The procedure was as in Example 1, but with 1.84 grams aluminium dihydrogen diethylenetriaminopenta-acetic acid (AlH₂-DTPA, 85.9 wt %, AKZO Nobel, 5 mmol) and 3.46 grams tetrabutylphosphonium hydroxide (TBPH, 40 wt % in H₂O, FLUKA, 5 mmol). The product was a viscous liquid at 120° C.

### EXAMPLE 9

The properties of a number of ionic liquids of the invention containing a chelating agent as anion were measured and are given in Table 1:

TABLE 1

| | | Water content | Viscosity at |
|---|---|---|---|
| | Physical form | by Karl Fisher | 50° C. |
| Compound | at 100° C. | (wt %) | (mPas) |
| GLDA-(TBA)₂ | Liquid | 0.5 | Not determined |
| GLDA-(TBA)₄ | Liquid | 2.4 | 65 000 |
| GLDA-(TBP)₂ | Liquid | 0.8 | Not determined |
| GLDA-(TBP)₄ | Liquid | 1.3 | 10 828 |
| GLDA-(choline)₄ | Liquid | 1.3 | Not determined |
| GLDA-(triethanol | Liquid | 8.5* | Not determined |
| amine)₄ | | | |
| DTPA-(TBA)₅ | Liquid | 3.0 | Not determined |
| DTPA-(TBP)₅ | Liquid | 2.2 | 19 946 |
| DTPA-(choline)₅ | Liquid | 5.0 | Not determined |
| DTPA-(tri ethanol | Liquid | 8.4* | 15 417 |
| amine)₅ | | | |
| HEDTA-(TBA)₃ | Liquid | 2.5 | 30 773 |
| HEDTA-(TBP)₃ | Liquid | 1.6 | Not determined |
| HEDTA-(choline)₃ | Liquid | 1.6 | 26 400 |
| HEDTA-(triethanol | Liquid | 2.0 | Not determined |
| amine)₃ | | | |

*decomposition during Karl Fisher analysis at 160° C.

### EXAMPLES 10-13 AND COMPARATIVE EXAMPLES 14 AND 15

The melting point of a number of ionic liquids was determined by subjecting them to a certain temperature and determining whether they were a liquid, a paste or a solid. A paste can be defined as a liquid with such a high viscosity that it stays in place when the jar is turned. The results are given in below Table 2.

TABLE 2

| (comp) | | 20° | 35° | 50° | 60° | |
|---|---|---|---|---|---|---|
| Ex. | Temperature | C. | C. | C. | C. | 70° |
| 10 | DTPA(TBA)₄H | Paste | Liquid | Liquid | Liquid | Liquid |
| 11 | GLDA(TBA)₃H | Paste | Paste | Paste | Paste | Paste |
| 12 | HEDTA(TBA)₂H | Paste | Liquid | Liquid | Liquid | Liquid |
| 13 | MGDA(TBA)₂H | Solid | Paste | Paste | Paste | Liquid |
| 14 | EDTA(TBA)₃H | Solid | Solid | Solid | Solid | Solid |
| 15 | NTA(TBA)₂H | Solid | Solid | Solid | Solid | Paste |

This example demonstrates that when using EDTA or NTA, a chelating agent in the acid form having a connectivity index ¹X/³X of higher than 2.17 and only one and the same monovalent cation besides a proton, the melting point is much higher than when DTPA, GLDA, HEDTA or MGDA is selected as the chelating agent.

### EXAMPLES 16-17

The melting point of a number of ionic liquids was determined by subjecting them to a certain temperature and determining whether they were a liquid, a paste or a solid. A paste can be defined as a liquid with such a high viscosity that it stays in place when the jar is turned. As the anion-contributing chelating agents were used TTHA (triethylenetetramine-N,N,N′,N″,N″′,N″′-hexaacetic acid), and CDTA (1,2-diaminocyclohexane-N,N,N′,N′-tetra-acetic acid). As the cation providing compound TBPH (tetrabutylphosphonium hydroxide) was used. The results are given in below Table 3.

TABLE 3

| Example | compound | T = 20° C. | T = 50° C. |
|---|---|---|---|
| 16 | TTHA-(TBP)₆ | Paste | Liquid |
| 17 | CDTA-(TBP)₄ | Paste | Liquid |

1. An ionic liquid of the formula:
(M^{m+})ₓH⁺_{y}nN⁺R₁R₂R₃R₄Z—N(—CHXCOO⁻)ₚY₂₋ₚ or
(M^{m+})ₓH⁺_{y}nP⁺R₁R₂R₃R₄Z—N(—CHXCOO⁻)ₚY₂₋ₚ
wherein
any one of R₁ to R₄ independently represents a hydrogen, alkyl, cycloalkyl, aryl or aralkyl group that may be unsubstituted or substituted with one or more groups selected from OH, Cl, Br, F, I, phenyl, NH₂, CN, NO₂, an ether group, COOR₅, CHO, COR₅ or OR₅, wherein R₅ is an alkyl or cycloalkyl group, and wherein two of the groups R₁ to R₄ together with the N atom may form a heteroaromatic or heterocyclic group;
Z—N(—CHXCOO⁻)ₚY₂₋ₚ is the anion derived from a chelating agent, wherein Z is a group selected from hydrogen, an alkyl group that may be unsubstituted or substituted with one or more carboxylate groups, hydroxyl groups and/or ether functionalities, or Z is a group of the formula —CH₂—CHR₆—R₇—N—(CH₂COO⁻)₂, R₆ is hydrogen or a C₁-C₃ alkyl group, R₇ is a single bond, a C₁-C₃ alkylene group or one or more groups of the formula —N(CH₂COO⁻)—CH₂—CHR₆—R₈—, R₈ is a single bond or a C₁-C₃ alkylene group, p is 2 or 1, X is H, an aryl group that may be unsubstituted or substituted with an OH and/or CH₃ substituent, or CH₂COO⁻, and when p is 1, Y is H, a benzyl group that may be unsubstituted or substituted with an OH and/or CH₃ group, or a phenyl group that may be unsubstituted or substituted with an OH and/or CH₃ substituent,
M^{m+} is a m-valent metal cation,
n is 1 or higher, x is from 0 to 5, y is from 0 to 3,
the total positive charge of (M^{m+})ₓ,H⁺_{y} and nN⁺R₁R₂R₃R₄ or nP⁺R₁R₂R₃R₄ equals the negative charge of the Z—N(—CHXCOO⁻)ₚY₂₋ₚ anion,
wherein at least one of the following conditions is fulfilled
(i) the chelating agent has a connectivity index ¹X/³X of below 2.17,
(ii) at least two different cations are present in the salt, whereby a proton cation does not count as one of the at least two,
(iii) at least one cation is a bivalent, trivalent, tetravalent or pentavalent cation, and having a melting point below 120° C.

2. The ionic liquid of claim 1 wherein the chelating agent of the formula Z—N(—CHXCOO⁻)ₚY₂₋ₚ (i) contains at least 1 chiral C atom, (ii) contains a nitrogen atom containing three different groups bound thereto, and/or (iii) contains three or more nitrogen atoms.

3. The ionic liquid of claim 1 wherein the anion Z—N(—CHXCOO⁻)ₚY₂₋ₚ is Z—N(—CH₂COO)₂.

4. The ionic liquid of claim 1 wherein the anion Z—N(—CHXCOO⁻)ₚY₂₋ₚ is selected from the group consisting of the anions of HEDTA (hydroxyethylethylenediamine triacetic acid), DTPA (diethylenetriamine penta-acetic acid), MGDA (methylglycine diacetic acid), and GLDA (glutamic acid diacetic acid).

5. The ionic liquid of claim 1 wherein the cation N⁺R₁R₂R₃R₄ or P⁺R₁R₂R₃R₄ is selected from the group consisting of the ammonium cations of C₁-C₄ alkyl- or C₁-C₄ alkanol-substituted amines, and the phosphonium cations of a C₁-C₄ alkyl- or C₁-C₄ alkanol-substituted phosphine.

6. The ionic liquid of claim 1 wherein y is from 0 to 1.

7. The ionic liquid of claim 1 wherein M^{m+} is selected from the group consisting of the cations of chromium, aluminum, copper, lithium, iron, zinc, nickel, titanium, and tin.

8. (canceled)

9. (canceled)

10. A method to prepare the ionic liquid of claim 1 comprising the steps of mixing a chelating agent or a (metal) salt thereof with an amine or phosphine or the salt, of an ammonium cation or phosphonium cation in a solvent and subsequently removing the solvent and the water or other compounds that are formed.

11. The method of claim 10 wherein the solvent is water.

12. The ionic liquid of claim 2 wherein the anion Z—N(—CHXCOO⁻)ₚY₂₋ₚ is Z—N(—CH₂COO⁻)₂.

13. The ionic liquid of claim 2 wherein the anion Z—N(—CHXCOO⁻)₂ is selected from the group consisting of the anions of HEDTA (hydroxyethylethylenediamine triacetic acid), DTPA (diethylenetriamine penta-acetic acid), MGDA (methylglycine diacetic acid), and GLDA (glutamic acid diacetic acid).

14. The ionic liquid of claim 4 wherein M^{m+} is selected from the group consisting of the cations of chromium, aluminum, copper, lithium, iron, zinc, nickel, titanium, and tin.

15. The ionic liquid of claim 5 wherein the cation N⁺R₁R₂R₃R₄ or P⁺R₁R₂R₃R₄ is selected from the group consisting of tetra-alkylammonium, trialkylammonium, trialkanolammonium, tetra-alkylphosphonium, trialkylphosphonium, and trialkanolphosphonium cations.

16. A method to apply the ionic liquid of claim 1 wherein both the beneficial properties of ionic liquids and the metal complexing or metal releasing properties of the chelating agent are used.

17. A reaction solvent, extraction solvent, electrolyte, catalyst, heat exchange fluid or coating composition comprising the ionic liquid of claim 1.

18. The method of claim 10 wherein the amine or phosphine salt is the hydroxyl salt, hydrogen carbonate (HCO₃⁻), methylcarbonate (CH₃OCOO⁻) or carbonate (CO₃²⁻) salt of an ammonium cation or phosphonium cation.

19. The ionic liquid of claim 1 wherein the heteroaromatic or heterocyclic group is a pyridinium, pyrrolidinium or imidazolium group.
